(19) United States
(12) Patent Application Publication
(10) Pub. No.: US 2026/0108151 A1
(43) Pub. Date: Apr. 23, 2026

(54) METHOD AND APPARATUS FOR FUNDUS IMAGING

(71) Applicant: PES Innovations, LLC, Fort Worth, TX (US)

(72) Inventors: Mark Darty, Frankllin, TN (US); Michael Paloian, Oyster Bay, NY (US)

(21) Appl. No.: 19/367,689

(22) Filed: Oct. 23, 2025

Related U.S. Application Data
(60) Provisional application No. 63/711,074, filed on Oct. 23, 2024.
(60) Provisional application No. 63/743,528, filed on Jan. 9, 2025.

(51) Int. Cl. A61B 3/12; A61B 3/15

(52) U.S. Cl. CPC A61B 3/12; A61B 3/158

(57) ABSTRACT

Disclosed are embodiments of a wide-field fundus imaging system integrating an optical head with multiple radial and central cameras, an illumination system with adjustable LED assemblies, and reflection control elements employing polarized filters to reduce glare. The system includes a handheld imaging device connected to external control and processing units via wired or wireless interfaces, with a disposable coupler for eyelid stabilization during imaging.

### CROSS REFERENCE TO RELATED APPLICATIONS

This application claims the benefit of the filing date of U.S. provisional patent application Ser. No. 63/711,074, filed on Oct. 23, 2024, entitled “METHOD AND APPARATUS FOR FUNDUS IMAGING,” and U.S. provisional patent application Ser. No. 63/743,528, filed on Jan. 9, 2025, entitled “METHOD AND APPARATUS FOR FUNDUS IMAGING,” the disclosures of which are incorporated herein by reference for all purposes.

### TECHNICAL FIELD

The invention relates in general to fundus cameras, and in particular to methods and devices for wide field of view (FOV) fundus imaging.

### BACKGROUND

A key cause of blindness in premature newborn children is Retinopathy of Prematurity (ROP). ROP is characterized by abnormal retinal vascularization, and it can lead to complications such as retinal detachment and partial or full blindness. For example, ROP is reported to be the disease that caused the popular singer Stevie Wonder's blindness. ROP affects children born prematurely. Each year more than 32,000 infants worldwide are diagnosed with ROP and it is among the most common causes of childhood blindness and reported incidence rates among premature newborns range from 10% to 30%.

According to the CDC in 2021, there were 3.6 million births in the USA of which it is estimated 10.5% (375,000) were premature. According to the World Health Organization (WHO data from 2020), approximately 130 million children were born worldwide, and using a 10.5% rate of prematurity, approximately 13.4 million children were born prematurely worldwide. All premature newborns are considered candidates for ROP screening.

Many cases of ROP related blindness can be treated with early detection, thereby saving many patients from unnecessary blindness. Early and ongoing diagnostic screening for ROP is considered essential in the first few weeks after premature birth and such screening is performed by Ophthalmologist examining patients with the use of retinal imaging systems called fundus cameras.

It is believed that existing fundus imaging cameras do not have a sufficient optical field of view to screen for ROP without physical manipulation of the eye by a highly skilled ophthalmologist. Furthermore, existing fundas imaging cameras used for ROP screen are relatively large and costly. Such devices and skilled ophthalmologists are mainly available in the developed world in hospitals that have neonatal intensive care units (NICUs) for premature newborns. In remote areas of the developed world and in almost all of the undeveloped world, such NICUs are not available and consequently ROP eye screening is not available or of limited availability. Consequently, there is a need for a new imaging device that has a wide field of view (i.e., exceeding 130 degrees) in a form factor that is highly portable to make it more effective in screening for the key diagnostic characteristics of ROP-especially in more remote regions that do not have a NICU in a local hospital.

### SUMMARY

In response to these and other problems, in one embodiment, there is a retinal imaging system with an ultra-wide field of view (exceeding 130 degrees) of the interior of the eye, as well as illumination and control electronics in a relatively compact, lightweight, and easy to use apparatus. The system may contain one or more image sensor devices, one or more optical lenses to capture and focus the image onto a photodiode array or imaging sensor. Certain embodiments may also employ additional optical components to aid in capturing extreme angles within the eye interior and/or polarizers and/or optical filters.

Certain embodiments may also be applicable to general eye imaging of not only premature newborn children, but all ages including older children, adolescents, adults, and the elderly. Such a system is also useful for imaging and diagnosing retinal disease in the elderly such as diabetic retinopathy and macular degeneration.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

FIG. 1 is a perspective illustration of one embodiment of a fundus imaging system comprising an external computer/display, a power communication module, a handheld imaging unit, and a coupler.

FIG. 2A is an isometric front view of the imaging unit of FIG. 1 showing the imaging unit and optical head.

FIG. 2B is an isometric back view of the imaging unit of FIG. 2A.

FIG. 3 is a conceptual functional diagram of certain components within an imaging unit, including the processor, memory, cameras, and LEDs.

FIG. 4 is an exploded illustration of the imaging unit's housing, illustrating certain internal components and the optical head.

FIG. 5A is a top view of one embodiment of a PCB assembly.

FIG. 5B is a bottom view of the PCB assembly of FIG. 5A.

FIG. 5C is another view of the PCB assembly and the optical head.

FIG. 6A is a front isometric view of the optical head.

FIG. 6B is a front isometric view of the optical head of FIG. 6A with certain diffusers and reflection control elements removed.

FIG. 6C is an isometric exploded view illustrating a chassis and camera array within the optical head.

FIG. 6D is a front view of one embodiment of a camera array with radial and center cameras.

FIG. 6E is a conceptual sectional view showing angular positioning of radial cameras relative to a center axis.

FIG. 6F is a conceptual section diagram illustrating cameras of the optical head positioned near an eye to show the effective field of view.

FIG. 6G is a conceptual illustration of a combined wider field of view merging images from multiple cameras.

FIG. 7A is an exploded isometric view of the chassis and certain illumination system components.

FIG. 7B is a front view illustrating an arrangement of outer and inner LED assemblies around the center axis.

FIG. 7C is a sectional view showing angles of LED assemblies with respect to the center axis.

FIG. 7D is a detailed isometric of an LED assembly.

FIG. 7E is an exploded isometric view of certain LED assembly components, including LED, diffuser, and lens.

FIG. 8 is an exploded isometric view of optical head, showing camera array and certain elements of the illumination system.

FIG. 9 is a front or proximal view of the camera array and certain portions of the LED assemblies, showing the relative placement of the cameras to the LED assemblies.

FIG. 10A is a perspective front or proximal view of the assembled optical head.

FIG. 10B is an exploded view of certain reflection control elements and the optical head.

FIG. 11 is a functional block diagram of certain software modules that may be incorporated in certain embodiments of the invention.

FIGS. 12A and 12B are portions of a flowchart illustrating the imaging process.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

Well-known elements are presented without detailed description in order not to obscure the present invention in unnecessary detail. For the most part, details unnecessary to obtain a complete understanding of the present invention have been omitted inasmuch as such details are within the skills of persons of ordinary skill in the relevant art. Details regarding control circuitry or mechanisms used to control the rotation of the various elements, such as mirrors and lenses, described herein are omitted, as such control circuits are within the skills of persons of ordinary skill in the relevant art.

When directions, such as upper, lower, top, bottom, clockwise, counter-clockwise, are discussed in this disclosure, such directions are meant to only supply reference directions for the illustrated figures and for orientation of components in the figures. The directions should not be read to imply actual directions used in any resulting invention or actual use. Under no circumstances, should such directions be read to limit or impart any meaning into the claims.

### System Overview

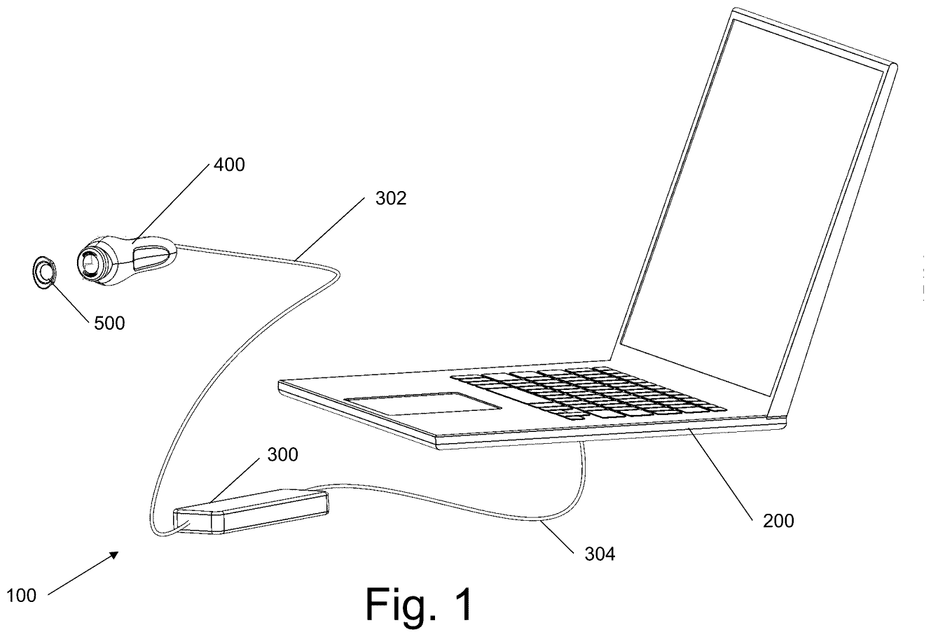

FIG. 1 is a perspective illustration of an example fundus imaging system 100. In certain embodiments, the fundus imaging system 100 comprises an external computer and/or display 200, a power communication module 300, a handheld imaging unit or camera 400, and a coupler 500.

In certain embodiments, the external computer 200 may be a laptop computer, phone, or tablet that includes some form of graphic display device. As is known in the art, the external computer contains a memory (not shown) with instructions for displaying a user interface for controlling the imaging unit 400, image processing and display, data presentation and recording any notes and other data associated with the imaging session.

In certain embodiments, embedded firmware or software running in a memory of the imaging unit 400 receives and implements commands from the external computer 200, captures one or more images from one or more cameras housed within the imaging unit for viewing one or more areas of the retina. As explained below, a processor within the imaging unit 400 multiplexes images from the cameras and sends the images as a data transmission to the external computer 200. In certain embodiments, the imaging unit 400 may communicate with and be powered by a power/communications cable, such as a USB cable (not shown), which is also removably coupled to the external computer 200. In other embodiments, the external power supply and interface module 300 supplies power and data to the imaging unit 400 through a standard USB interface and/or cable 302. In yet other embodiments, the power supply and interface module 300 may use a custom-designed cable 302 to provide both power and communication to the imaging unit 400. In certain embodiments, the module 300 may couple to an AC power cord (not shown) for coupling to an AC electrical wall plug (not shown). In certain embodiments, the module 300 also couples to a second communication cable 304, such as a USB cable, which allows communication, data, and/or power transfer between the module 300 and the imaging unit 400. In yet other embodiments, the external computer 200 may communicate with the imaging unit 400 by wireless methods wherein the imaging device 400 may be battery operated, such that the cable 302 and power/communications module 300 are unnecessary.

Thus, depending on the specific embodiment and power requirements, the power sources for the imaging unit 400 may be the external computer 200, the power supply and interface module 300, or an internal battery (i.e., rechargeable or replaceable) housed within the imaging unit 400 enclosure.

In certain embodiments, the coupler 500 is disposable and designed to couple to the imaging unit 400 wherein the coupler 500 would slide under the patient's eyelid to keep the eyelids open during imaging. In certain embodiments, the coupler 500 may be made from a sterilized soft hygroscopic polymer, similar to a material used for contact lenses. In other embodiments, the coupler may be formed from a semi-rigid material that is not hydroscopic. In certain embodiments, the coupler 500 is molded with a circular opening and an adapter ring to mate with the distal end of the imaging unit 400. In certain embodiments, the coupler 500 also positions the imaging unit 400 at an optimum location with respect to the patient's eye for obtaining images. In addition to assisting with the proper positioning of the imaging unit 400 with respect to the patient's eye, the coupler 500 may provide sterility and protection for the patient's eye during the imaging session. Once the imaging session is complete, the coupler 500 may be detached from the reusable imaging unit 400 and disposed of according to standard clinical procedures for items that come in contact with patient's anatomy.

For more information on one embodiment of the coupler 500, see the Applicant's provisional patent application 63/846,266, filed on Jul. 18, 2025 entitled, “METHOD AND APPARATUS FOR FUNDUS IMAGING,” the enclosure of which is hereby incorporated by reference for all purposes.

### The Imaging Unit

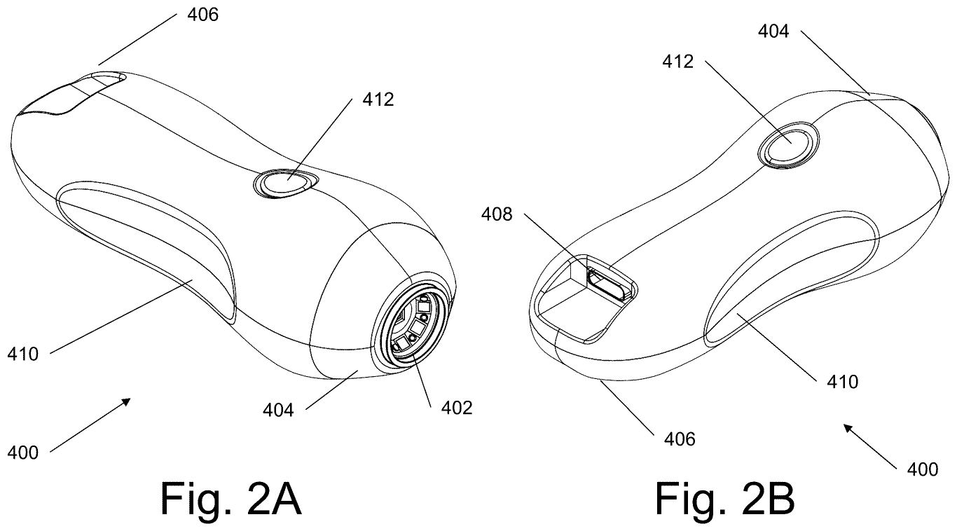

FIG. 2A is an isometric drawing taken from the front or distal perspective illustrating one embodiment of the imaging unit 400. FIG. 2B is an isometric drawing taken from the back view or proximal view of the imaging unit 400. As can best be seen in FIG. 2A, an imaging and illumination head (imaging head) 402 is positioned at a distal end 404 of the imaging unit 400. In certain embodiments, at a proximal or opposing end 406 of the unit 400 is a port for a power and/or data cable, such as a communications and/or USB interface 408. An ergometric and contoured housing 410 encloses the components of the imaging unit 400 and is designed for handheld orientation in both pointing and image capture positions.

In certain embodiments, the imaging unit 400 may have a user interface, such as an interface or control button 412. For example, in one embodiment, pressing and holding the control button 412 for a short period of time will turn the imaging unit 400 on or off. Pressing the control button 412 for shorter period of time starts a video recording process to facilitate positioning. A rapid push and release of the control button 412 may initiate the capture of a still image.

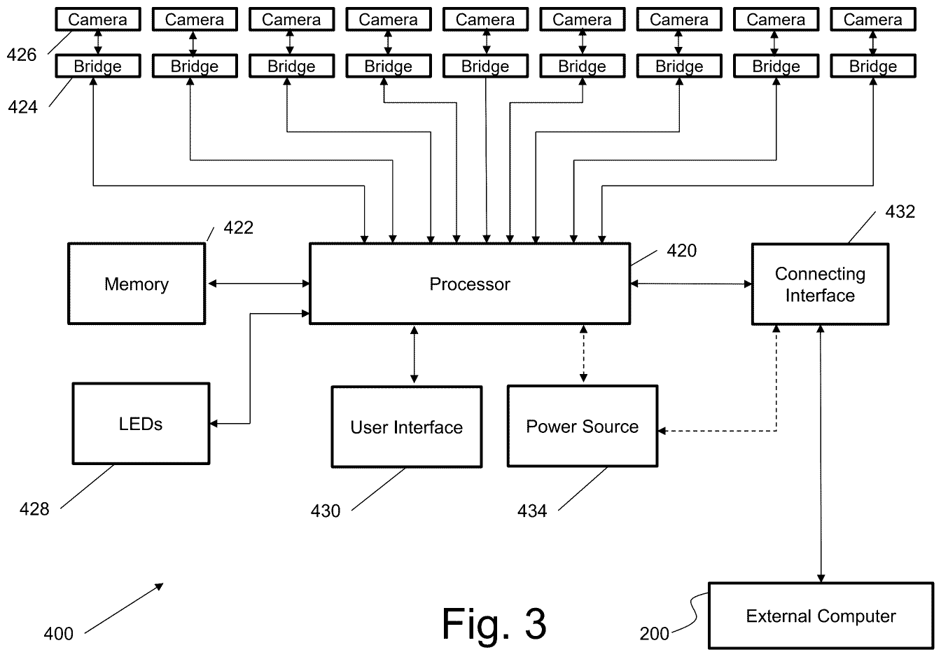

FIG. 3 is a conceptual functional diagram of certain components of the imaging unit 400. In certain embodiments, the imaging unit 400 includes a processor 420 for controlling the overall functions of the system. The processor 420 is coupled to a memory 422 for storing programming instructions via firmware and/or software. In certain embodiments, the processor 420 may be a programmable logic chip with a 600 mhz computational core and 256 mb of RAM. In certain embodiments, a System-on-Chip (SoC) processor may be used—for example, a Zynq-7000 XC7Z045 processor from Xilinx, which is a dual-core ARM Cortex-A9 processor, operating at up to 1 GHz (or preferably at 600 GHz). Such an example processor may offer a high amount of logic cells, DSP slices, and block RAM, and have memory interfaces to support DDR3, DDR3L, DDR2, and LPDDR2 memories. Such a processor may have peripheral support for interfacing with USB, Ethernet, UART, SPI, I2C, and other standard interfaces.

In certain embodiments, the processor 420 is also in communication with a plurality of interface chips or bridge processors 424, which correspond with and control a plurality of cameras, camera modules or camera sensors 426. In certain embodiments, the camera bridge processor 424 may be a camera bridge processor that can provide an integrated analog to digital conversion and certain ISP functionality including Auto White Balance (AWB), Auto Gain Control (AGC) and Auto Exposure Control (AEC), Staggered HDR and multi-frame HDR. In certain embodiments, the camera bridge processor can accept 4-wire digital or analog input and MIPI input and convert it to DVP or MIPI output for signal processing. While converting the signal, the camera bridge processor may also provide image clean-up, image resizing, and manipulations, including frame rate control, mirroring, and flipping. During the operation, input signals from the image sensors of the cameras are digitized by an ADC and then processed by a digital signal processor (DSP) and finally standard MIPI/DVP outputs are sent out. One such example of a camera bridge processor is the camera bridge processor OAH0428 from Omnivision.

In certain embodiments, the plurality of cameras or camera array 426 may include endoscopic cameras or camera chips containing built in fixed focal length optical lens with a wide field of view and a relatively wide depth of field to avoid the need for additional focusing mechanisms or more complex camera lens. For instance, in one embodiment, a camera module including an image sensor with dimensions of no more than 3 mm×3 mm×5 mm (and preferably 2.5 mm×2.5 mm×4.5mm) may be used. Such a camera module may have a 5.5 f-stop which allows for a depth of field of 8 mm to 100 mm—reducing the need for a separate focusing system. One example camera or camera chip that may be used in certain embodiments is the Omnivision OCH2B10 2 megapixel CameraCubeChip—which has a 1/7.5 CMOS 2 Megapixel sensor and is available in a small form factor of 2.5×2.5 mm.

An appropriate quantity and quality of light illumination may be achieved by utilizing LED lens, diffusers, polarizers, electronic control circuits, and software, etc. such that the interior of the patient's eye is illuminated with a uniform amount of light that is both safe for the retina and allows sufficient photographic illumination that the imaging unit 400 can capture adequate images of the interior of the eye. In one embodiment, a plurality of LEDs 428 is provided and is controlled by the processor 400. In such an embodiment, illumination is produced, for example, by LED sources, and said illumination enters the pupil of the subject's eye, thus illuminating the interior of the eye. The details of one embodiment of a plurality of LEDs 428 are discussed below. In other embodiments, ring-like lightwells, mirrors, and/or light channels may be used in conjunction with one or more LEDs to provide adequate illumination.

As discussed previously, a user interface 430, such as the control button 412 (see FIGS. 2A and 2B), allows the user to have basic control functions over the imaging unit 400. In other embodiments, the user interface 430 may include multiple buttons, a status indicator such as an LED, and/or a microphone for receiving voice commands and/or recording notes from the user.

The processor 420 is also in communication with a connecting interface 432, which allows communication with the external devices, such as the external computer 200 as explained above. In certain embodiments, the connecting interface 432 may include USB, Ethernet, WIFI, Bluetooth, or similar technologies used to interface with computers and networks. Thus, images produced by certain embodiments may be uploaded to local and wide area networks via data interfaces with those networks. Once the images are available to these networks, they may be viewed remotely and stored in storage devices coupled to such networks. Thus, in certain embodiments, the analysis and diagnostics of certain diseases may take place remotely via telemedicine.

As discussed above, a power source 434 provides electrical power to the processor 420 and other necessary components such as the memory 422 and the LEDs 428—either directly, if an internal battery is provided, or through the connecting interface 432 in embodiments without an internal battery. In embodiments where an internal battery is provided, the internal battery may be in electrical communication with a charging circuit (not shown), which may receive power via the connecting interface 432 and a custom cable that provides independent power and data transfer capabilities. In other embodiments, standard USB cables may be used. In yet other embodiments, there may be an independent charging circuit (not shown) for inductively charging the internal battery.

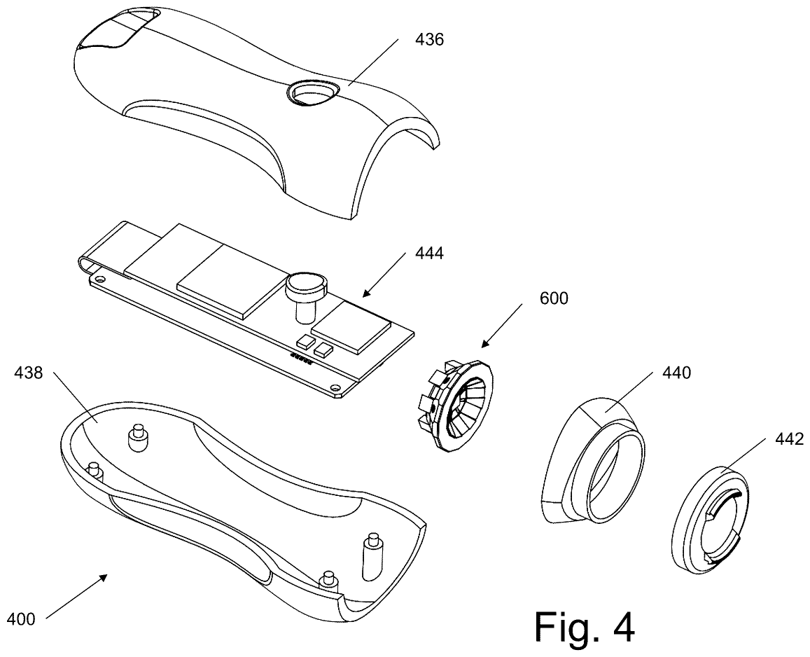

FIG. 4 is an isometric exploded illustration of various components of an embodiment of the imaging unit 400. In the illustrated embodiment, there is a top cover or enclosure 436, a bottom enclosure 438, a front or distal end enclosure 440, and a clear cover 442 designed to mate with the coupler 500 discussed above. The various enclosures and covers 436 through 442 house a printed circuit board (“PCB”) assembly 444 and the optical head 600.

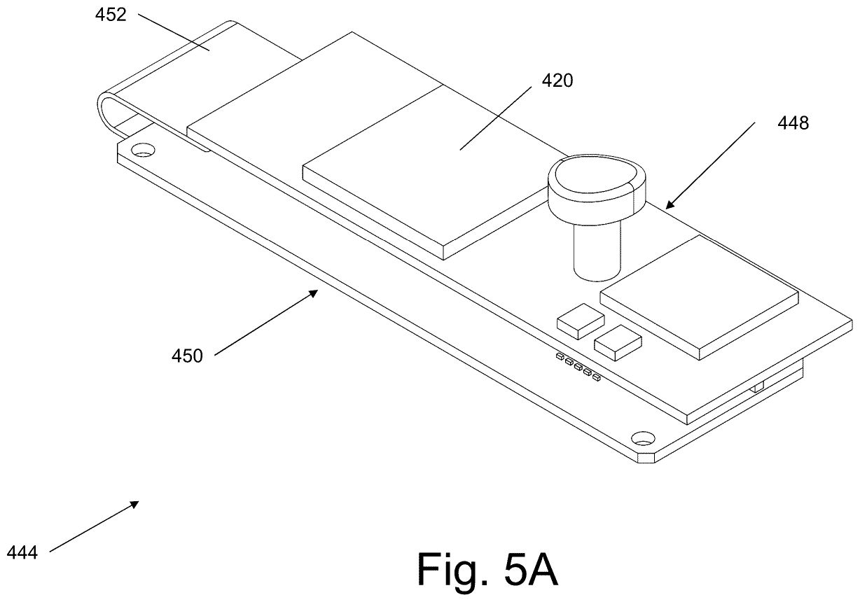

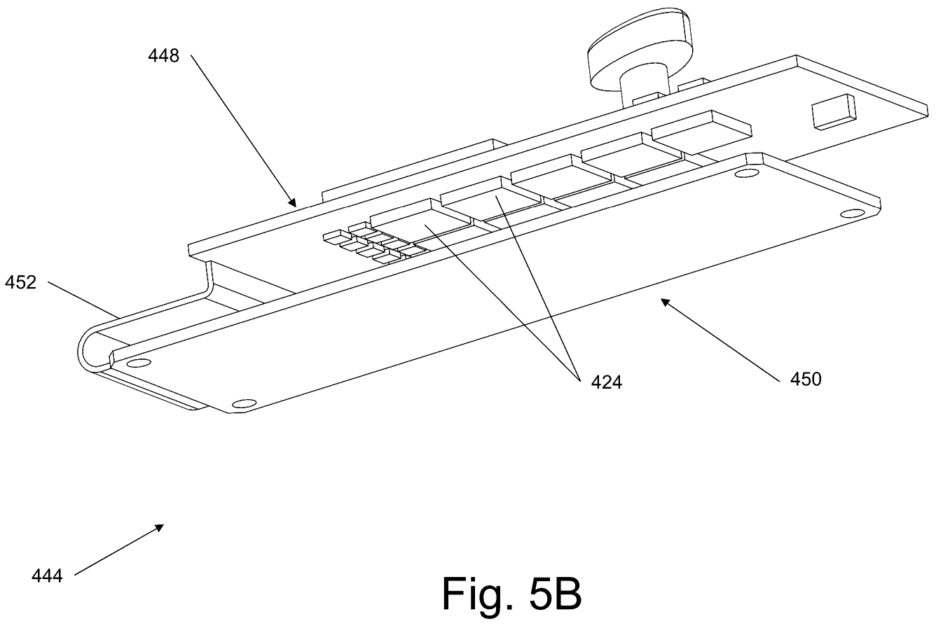

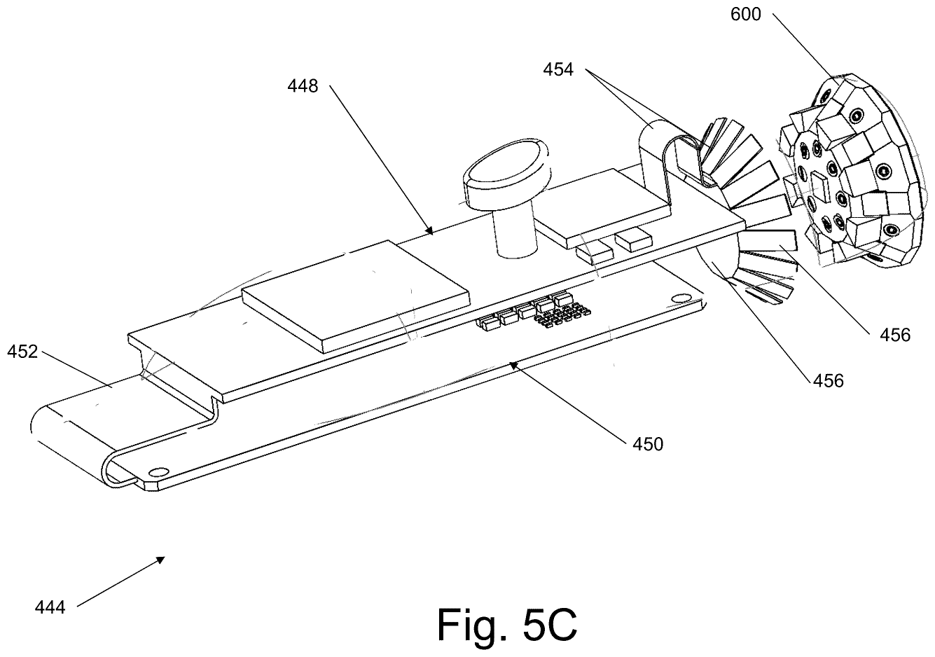

As discussed above, the top enclosure 436, the bottom enclosure 438, and the front or proximal enclosure 440 constitute one embodiment of the housing 410 and may be made of medical grade polymer materials which can be readily sterilized with alcohol or another disinfectant used to clean and disinfect similar medical devices. In certain embodiments, the clear cover 442 may be formed from clear glass, polymer or a combination of both for the purpose of protecting the internal imaging parts of the device 400 and also to provide a surface that can be cleaned and disinfected. In some embodiments the shape of the optical transparent cover within 442 may be specially shaped to conform to the shape of the eye or cornea such that the overall imaging unit 400 may be positioned as close as possible to the eye cornea for the purposes of gaining the widest field of view through a dilated or undilated pupil. Turning now to FIG. 5A, there is a top isometric view of one example embodiment of the PCB assembly 444, which includes various electronic components that may be implemented in certain embodiments. In contrast, FIG. 5B is a bottom perspective view of the PCB assembly 444 and certain electrical components illustrated in FIG. 5A. In the example embodiment illustrated in FIGS. 5A and 5B, there is a top or data PCB 448, which is coupled to various components for data processing, camera management, and LED control, and a lower or power management PCB 450, which is connected to various components for controlling power. In certain embodiments, the PCB 448 and PCB 450 communicate and distribute power via a ribbon cable or flexible printed circuit 452.

In the example embodiment illustrated in FIGS. 5A and 5B, the processor 420 (FIG. 3) may be positioned on the data PCB 448. In certain embodiments, the individual camera bridge chips 424 may also be positioned on the PCB 448. In the illustrated example, the bridge chips 424 are positioned on the PCB 448 on a side opposing the processor 420 to keep the footprint of the PCB small to fit within a handheld enclosure 410 (See FIG. 4). In embodiments using a predetermined number of camera or camera sensors 426 (e.g., nine), there may be a corresponding number of camera bridge chips 424 positioned on the PCB 448 as indicated in FIG. 5B.

In certain embodiments, a combination of flexible printed circuits 454 and/or rigid circuit boards 456 may be used as an interface between the main PCBs 448 and 450 and the camera sensors 426 and the LEDs 428 positioned within the optical head 600 as illustrated in FIG. 5C. Thus, communication and power may be distributed from the PCBs 448 and 450 to the electrical components of the optical head 600. In yet other embodiments, communication and power may be distributed from the PCBs 448 and 450 to the optical head 600 using conventional ribbon wire or other connections known in the art.

### The Optical Head

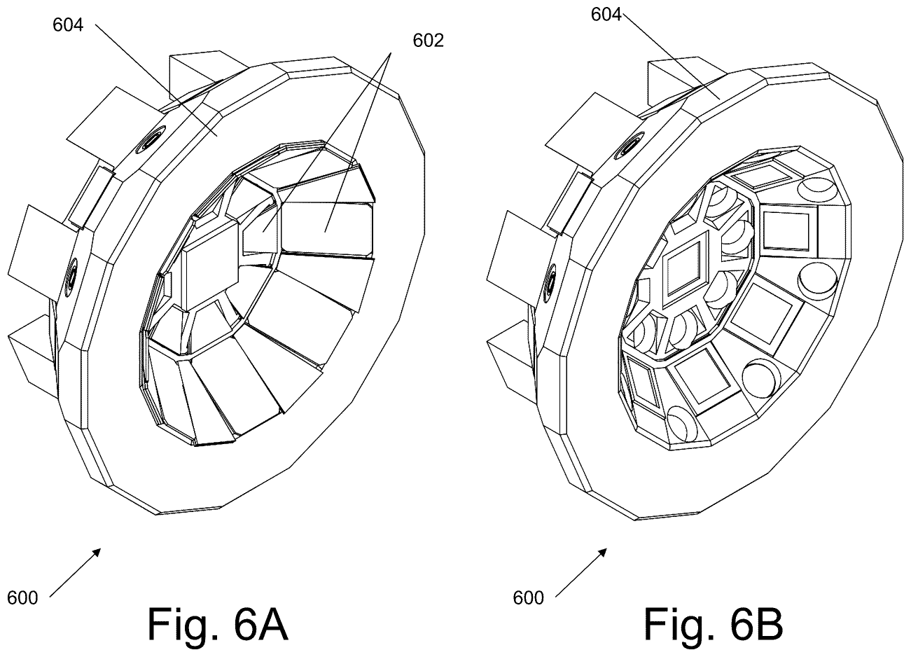

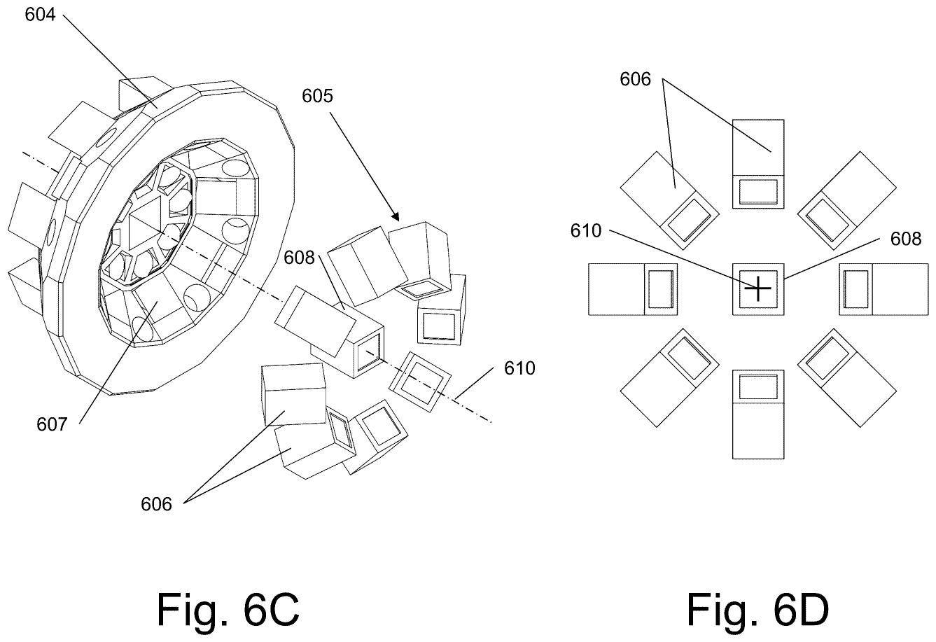

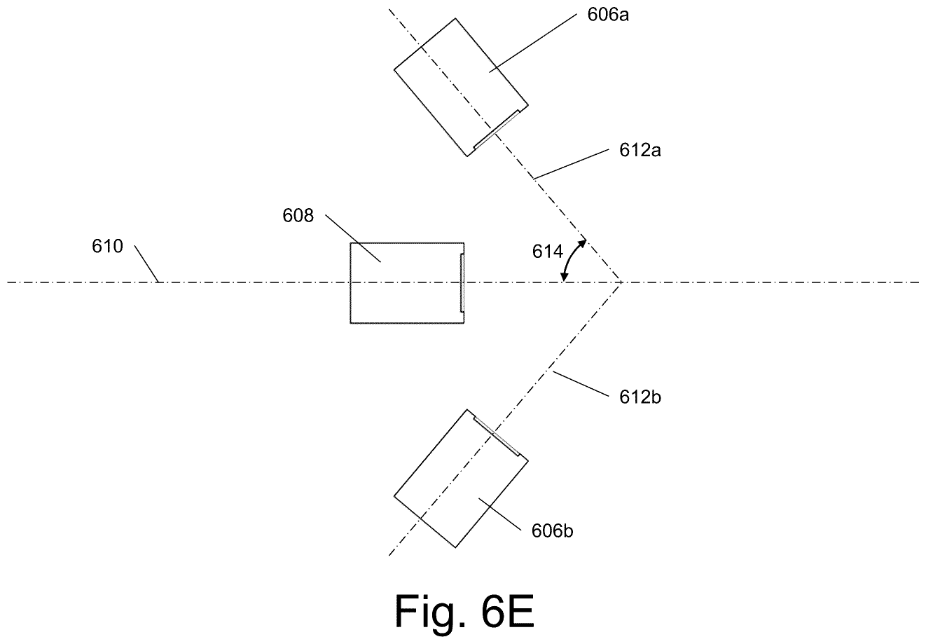

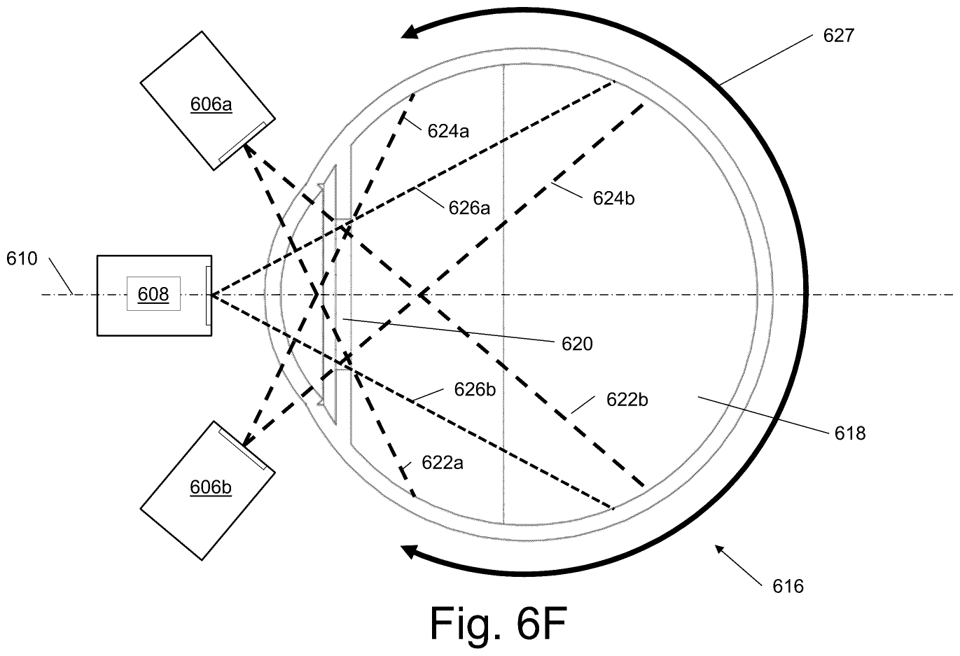

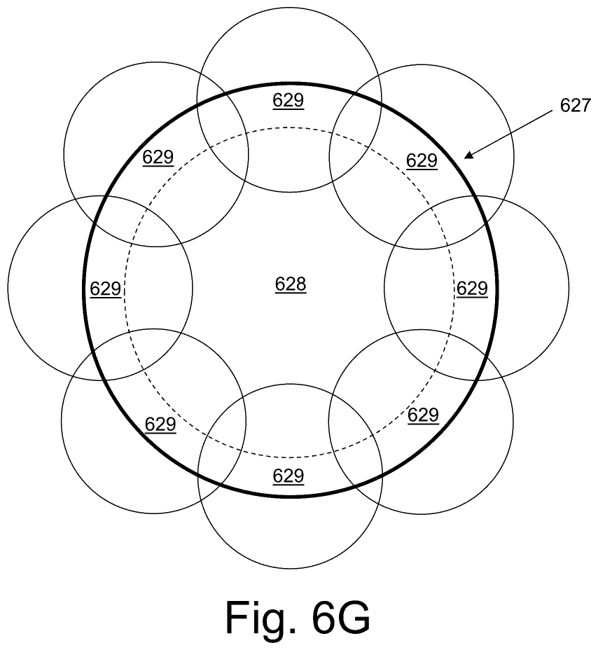

FIG. 6A is a front or distal isometric view of one embodiment of the optical head 600. FIG. 6B is a distal isometric view of the optical head 600 with certain reflection control elements 602 removed for discussion purposes. FIG. 6C is an exploded view illustrating a chassis 604 and a camera array 605. FIG. 6D is a front or distal view of one embodiment of the camera array 605.

The chassis 604 is designed to position and secure the camera array 605 around a main or center longitudinal axis 610 of the camera head 600. In certain embodiments, the chassis 604 is formed from single piece of rigid material, such as polycarbonate, machined aluminum, or another similar material to position and secure the individual cameras in the camera array 605 at precise locations and angles. In order to accomplish this positioning, there are notches or apertures 607 formed at precise angles within the chassis 604 which are designed to receive and position the individual cameras forming the camera array 605.

In certain embodiments, the camera array 605 comprises a plurality of radial cameras 606 angularly positioned around the center axis 610 as indicated in FIG. 6D. For instance, in the illustrated embodiment of FIGS. 6C and 6D, there are eight equally spaced radial cameras or camera chips 606 angularly positioned, via the chassis 604, around the longitudinal axis 610. Additionally, in the illustrative embodiment, there is a center camera 608 centered upon and aligned with the longitudinal axis 610. In certain embodiments, the eight radial cameras 606 and the center camera 608 are the cameras or camera chips 426 discussed above in reference to FIG. 3.

FIG. 6E is a conceptual section view parallel to the center axis 610 showing the relative angular position of two of the radial cameras 606a and 606b, and the center camera 608, with respect to the center axis 610. As illustrated in FIG. 6E, the radial camera 606a has its own longitudinal axis 612a. Similarly, the radial camera 606b has its own longitudinal axis 612b. An angle of intersection 614 is the angle at which the center axis 610 intersects with the longitudinal axes 612a and 612b of the individual radial cameras 606a and 606b, respectively. For instance, as illustrated in FIG. 6E, the longitudinal axis of the radial camera 606a intersects with the center axis 610 at an angle 614. In certain applications, for instance, in neo-natal patient applications, a preferred angle of intersection is 50 degrees and is designed to obtain a field of view that includes the full retina including the boundary with the ora serrata. In other embodiments, the angle of intersection 614 could be within a range corresponding to the range of eyeball sizes from neonatal to adult patients, such as 35 to 65 degrees. The preferred angle of intersection 614 may depend on the relative size of the patient's eye with respect to the overall positioning of the radial cameras 606 and the specific medical use application of the camera system 100. However, in yet other embodiments, a single angle of intersection 614 may be used, but the overall field of view of a patient's retina may be changed based on the relative position of the radial cameras with respect to the size of the patient's eye.

FIG. 6F is a not-to-scale conceptual illustration of one embodiment of the three cameras of the optical head 600 positioned adjacent to a patient's eye 616. In FIG. 6F, the center camera 608 and the radial cameras 606a and 606b are angularly aligned with the longitudinal axis 610 in a manner similar to the illustrative embodiment of FIGS. 6D and 6E. Although the cameras 606a, 606b, and 608 may have a wide “native” field of view, their “effective” field of view of the patient's retina 618 is obscured by the patient's pupil opening 620, whether dilated or non-dilated. For instance, the effective field of view of the radial camera 606a can be represented by the dashed lines 622a and 622b. Similarly, the effective field of view of the radial camera 606b can be represented by the dashed lines 624a and 624b. The effective field of view of the patient's retina generated by the center camera 608 is also limited by the patient's iris-but to a lesser degree than the radial cameras, as indicated by the dotted lines 626a and 626b. FIG. 6F illustrates the utility of the combined overlapping imaging from the multiplicity of cameras positioned in the geometry discussed, so as to obtain imaging detail of all areas of the retina to the ora serrata which requires a total field of view exceeding 180 degrees, which the applicant does not be believe is achievable with a single prior art camera and wide field of view lens. In fact, if the field of view 627 is conventionally measured from the center of the eye, in certain embodiments, the field of view from the combined cameras approaches 228 degrees for some patients—which is up to and including the ora serrata.

As will be discussed in detail later, in some embodiments, combining the individual images produced from the eight radial cameras 606 and the center camera 608 provides a merged image having roughly up to a 228-degree field of view of the retina (depending on the size of the patient's eye) as conceptually indicated by the not-to-scale illustration of FIG. 6G. FIG. 6G illustrates the larger field of view 628 possible when the effective field of view of the center camera 608 and each of the eight peripheral field of views 629 of the radial cameras 606 are combined. Such a wide field of view allows for an image of the retina to be generated up to and including the ora serrata when the camera optical head is positioned adjacent to a patient's eye with or without the coupler 500 as discussed above.

### Illumination System

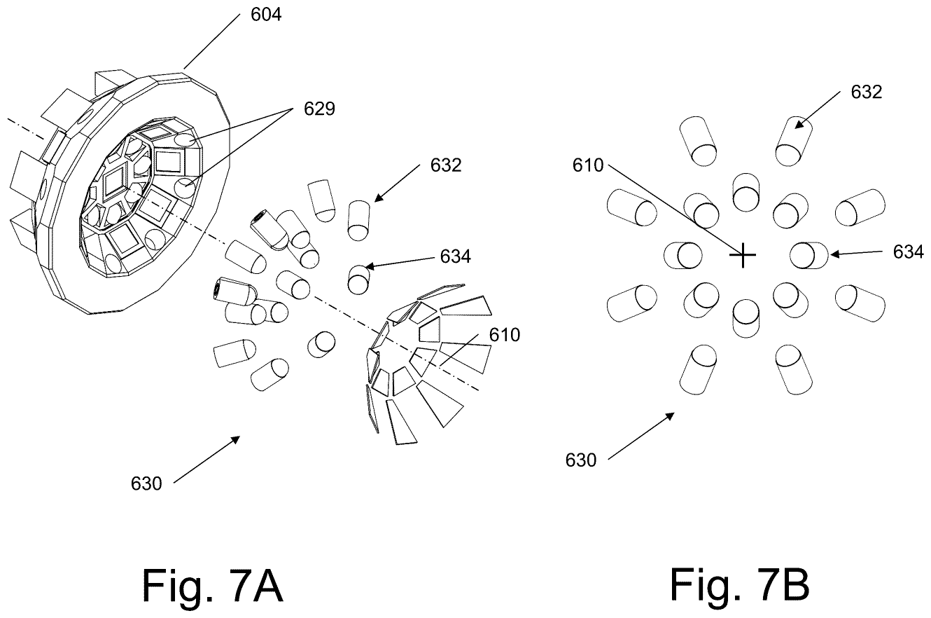

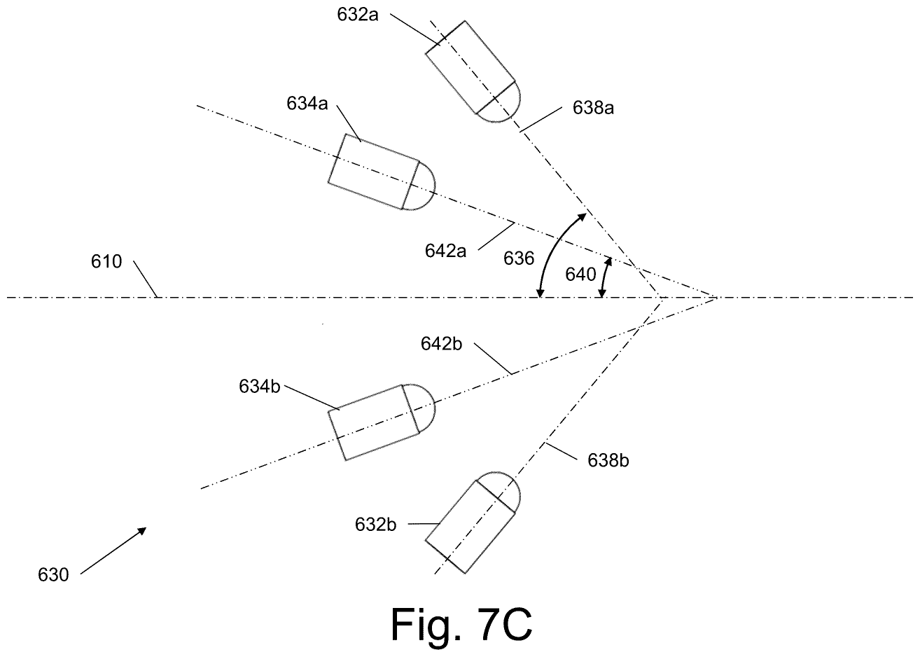

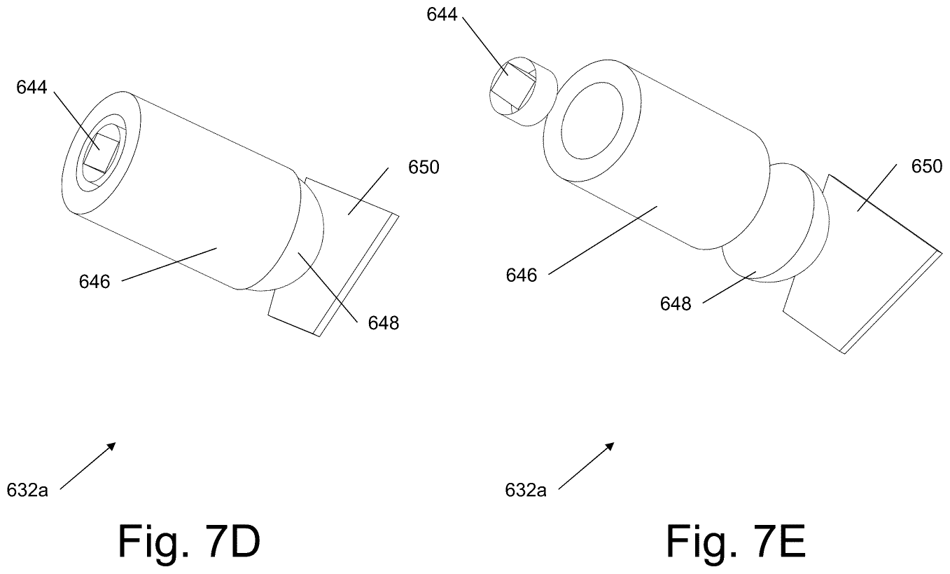

In certain embodiments, an illumination system is provided to illuminate the retina to assist with the generation of usable images. FIG. 7A is an exploded isometric view of the chassis 604 and one embodiment of an illumination system 630 that is illustrated positioned away from the chassis 604 for discussion purposes. As discussed above, in addition to notches or apertures for the camera array, in certain embodiments, the chassis 604 has a plurality of apertures 629 for positioning and securing individual elements of the illumination system 630.

When the camera head 600 is assembled, therefore, the illumination system 630 is positioned and secured in place inside the chassis 604 in such a manner as to allow uniform lighting of the interior surface of the eye. In certain embodiments, the illumination system 630 comprises a first or outer plurality of radial LED assemblies 632 angularly positioned around the center axis 610 as indicated in FIG. 7B. For instance, in the illustrated embodiment of FIG. 7B, there are eight equally spaced LED assemblies 632 angularly positioned around the longitudinal axis 610. Additionally, in the illustrative embodiment, there is a second or inner plurality of eight LED assemblies 634, which are also centered around the longitudinal axis 610 as illustrated in FIG. 7B.

FIG. 7C is a conceptual section view parallel to the center axis 610 showing the relative angular position of two of the outer plurality of LED assemblies 632a and 632b, and two of the inner plurality of LED assemblies 634a and 634b, with respect to the center axis 610. An angle of intersection 636 is the angle at which the center axis 610 intersects with the longitudinal axes 638a and 638b of the individual outer LED assemblies 632a and 632b, respectively. For instance, as illustrated in FIG. 7C, the longitudinal axis of the outer LED assembly 632a intersects with the center axis 610 at an angle 636, which in some embodiments is 50 degrees. Similarly, an angle of intersection 640 is the angle at which the center axis 610 intersects with the longitudinal axes 642a and 642b of the individual inner LED assemblies 634a and 634b, respectively, which in certain embodiments is 20 degrees. However, the exact angle of intersections 636 and 640 will depend on the specific application and size of the eye to be imaged-which as discussed above, may be determined by the angle of intersection 614 of the cameras 606 in the camera array 605, which might vary as much as 15 degrees in either direction.

In certain applications, for instance, in neonatal patient applications, a preferred angle 636 of intersection is 50 degrees. but the angle of intersection 636 could be within a range of to allow the design to accommodate various sizes of eyeballs ranging from neonatal to adults, such as between 35 and 65 degrees. The preferred angle of intersection 636 may depend on the relative size of the patient's eye with respect to the overall positioning of the radial cameras 606 and the specific medical use application of the camera system 100 as explained above. Similarly, in certain applications, a preferred angle 640 of intersection is 50 degrees but the angle of intersection 640 could be within a range corresponding to different eyeball sizes from neonatal to adult patients. In certain embodiments the LED assemblies will include, but not be limited to, some or all of the following parts such as a light emitting diode source, a light pipe to transfer the light from the proximally located source to the distal end of the pipe, a lens to orient and disperse the light onto the target, a diffuser to spread the light so as to be more homogenous, and a polarizer constructed and positioned such that when crossed appropriately relative to other polarizers positioned over the cameras, reflections are minimized as seen by the one or more cameras.

FIG. 7D is a detailed isometric illustration of one embodiment of an LED assembly 632a, which may be used in various embodiments of the illumination system 630 as discussed above. FIG. 7E is a detailed exploded isometric illustration of the LED assembly 632a, illustrating the major components of the LED assembly 632a. As illustrated, the LED assembly 632a comprises an individual LED 644 positioned at the proximal end of a light tube 646. The individual LEDs 644 are one embodiment of the LEDs 428 discussed above in reference to FIG. 3. In one embodiment, when combined with polarizers or other reflection control systems, the individual LEDs 644 may have an output of 690 millicandella and a field of view of 120 degrees, but such light intensity and field of view of the projected light from the LED could vary within this embodiment and other embodiments. One such example LED that may be used in certain embodiments is a surface mount device (SMD) printed circuit board (PCB) type LED (size no. 0402) available from Inolux.

A lens 648 is positioned at the distal end of the light tube 646 to capture and transfer the light produced by the LED to the distal end of the LED assembly 632. On the distal end of the LED could be stacked or integrated a diffuser 650 and lens 648 wherein the diffuser has a purpose of making the light more homogenous and better distributed, and the lens has a purpose of projecting the light more uniformly onto the target areas within the eye. Thus, in the illustrative and example embodiments, there are two groups of eight LED assemblies forming the illumination system 630, which means there are a total of 16 LEDs, 16 Light tubes, 16 lenses, and 16 diffusers comprising the illumination system.

In alternative embodiments, the LED assemblies 632 may be replaced by LEDs positioned at the proximal end of a borehole drilled or formed through the chassis 604 which would then allow the light from the LED to exit at the distal end of the bore hole. In such embodiments, the interior of the borehole forms a light pipe which directs the light. In yet another embodiment, a reflective metallic hollow pipe or a transparent glass or polymer rod could be inserted into the bore for purposes of transferring light emitted from the LEDs to the distal end of the LED 644 assembly 632a. In yet other embodiments, ring lighting systems and or lightwells may be used to produce the required amount of illumination.

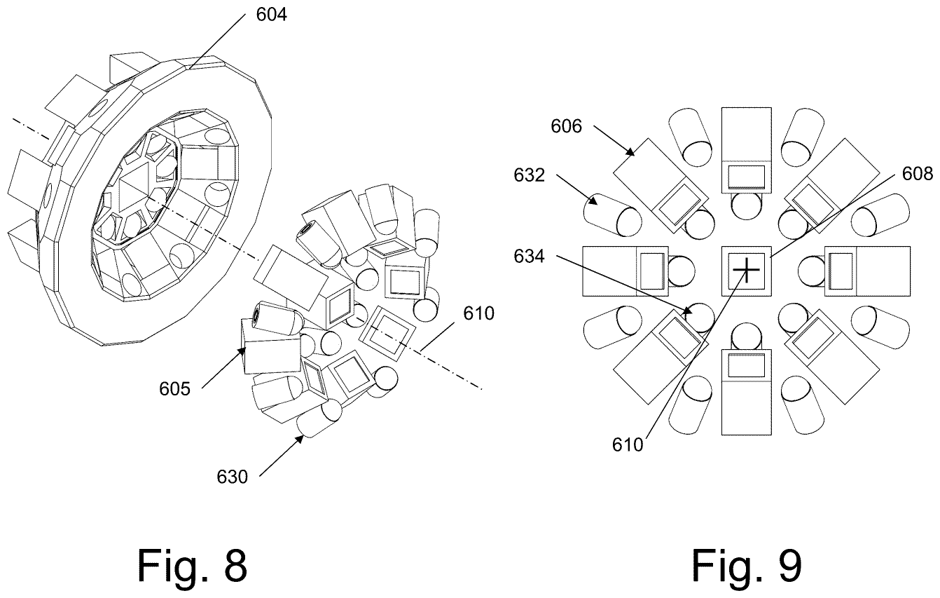

FIG. 8 is an exploded isometric view of the optical head 600, illustrating the chassis 604, the camera array 605, and portions of the illumination system 630 where the camera array 605 and the illumination system 630 are positioned away from the chassis 604. As discussed above, when the camera head 600 is assembled, the camera array 605 and the illumination system 630 will be positioned and secured in place inside the chassis 604. FIG. 9 is a proximal end view of the camera array 605 and a portion of the illumination system 630, which is illustrated as normal to the center axis 610. FIGS. 8 and 9 illustrate the relative positioning of the camera array 605 to the illumination system 630. As illustrated in FIGS. 8 and 9, the plurality of outer LED assemblies 632 are interspersed between the plurality of radial cameras 606 and are positioned to provide illumination to the effective field of view of the radial cameras 606. With respect to the center axis 610, the plurality of inner LED assemblies 634 are radially positioned below the radial cameras 606 and are positioned to provide illumination to the effective field of view of the center camera 608.

### Reflection Control System

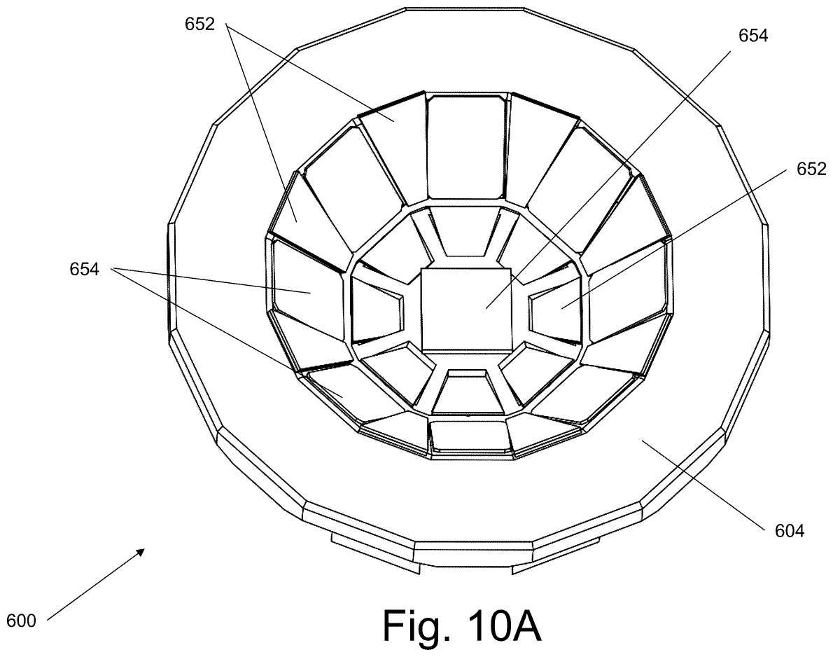

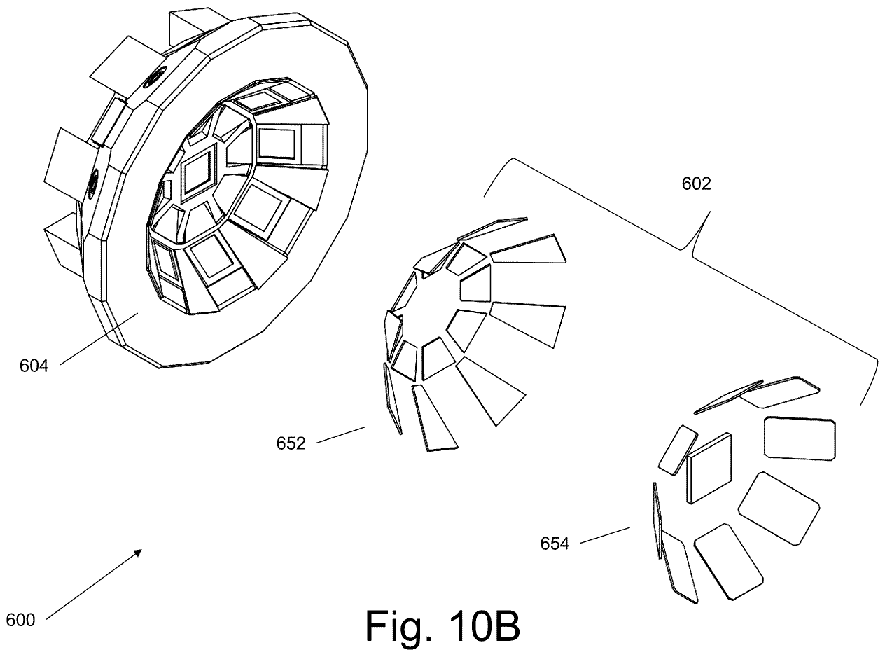

FIG. 10A is a perspective drawing illustrating the distal view of one embodiment of the assembled imaging head 600. In contrast, FIG. 10B is an exploded isometric drawing of the imaging head 600 with certain reflection control elements 602 positioned away from the chassis 604 for discussion purposes.

As illustrated in FIGS. 10A and 10B, in certain embodiments, the reflection control elements 602 may be a plurality of diffusers 650 (not shown in FIG. 10A) which are sized and positioned over the LED assemblies 632 and 634, a plurality of polarizing filters 652 which are sized and positioned to fit over or under the diffusers 650, and a plurality of polarizing filters 654 sized and positioned to fit over each camera or camera chip in the camera array 605, such that the LED assembly polarizers are crossed with the Camera polarizers 654 so that only light within cross polarization will reach the camera after said light has interacted with and been reflected from the target retina area within the eye. By use of linear polarizers that are crossed 90 degrees, the system will reject light reflections and pass light that is properly polarized within alignment. In certain embodiments, the polarizing material may be a flexible film affixed into position and in others it could be polarization implemented on glass wherein the glass is cut and fitted into the assembly, such polarizers on glass, wherein such polarizers are positioned using a robotic pick and placement automated process due to the small size of the filters.

Using polarizing filters or films 652 helps reduce reflections when used with the polarizing filter or film 654 positioned in “front” of the cameras in the camera array 605 by controlling the polarization state of the light before it reaches the camera sensors. By placing a polarizer directly over the LED illumination system and then placing another polarizer over the camera, wherein the two polarizers are oriented 90 degrees from each other, unwanted reflections can be reduced or attenuated. This approach effectively increases the optical signal-to-noise ratio, but at the expense of a loss of overall light reaching the camera. It is the LED polarized light that interacts with the tissue inside the eye and backscatters to the camera sensor that we are allowing to pass from LED back to the camera. The camera's polarizing filter 654 is oriented to accept the polarized light that interacts with the target but to block or attenuate unwanted light reflections from non-target areas such as the camera cover, the cornea, the organic eye lens or the surface of the retina because such reflected and bounced light arrives at the camera from angles and orientations other than a direct line between the LED and the camera. By orienting the LED and camera polarizers as close to 90 degrees apart relative to one another, the unwanted amount of reflected light from surfaces other than the target is significantly reduced. This results in images with fewer glare and reflections, improving clarity and contrast.

### Software and Operation

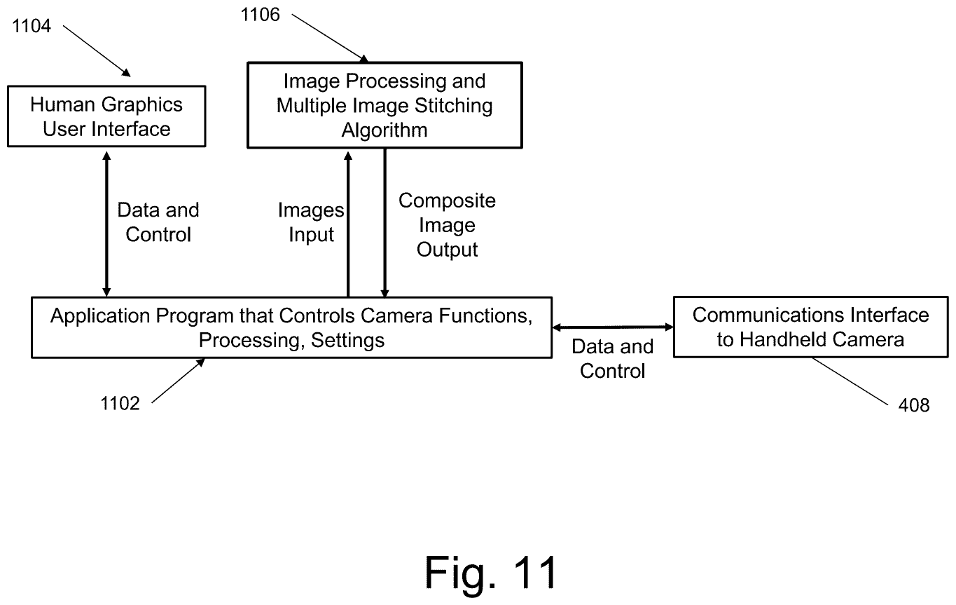

FIG. 11 is a functional block diagram of certain software functions and/or modules of one embodiment that may be implemented in the imaging system 100. Such software modules and/or algorithms may be stored and executed in the various computer memories described herein. In certain embodiments, there may be a main or primary application program 1102, a graphics user interface (“GUI”) module 1104, an image processing module or algorithm 1106 which, in certain embodiments, is executed on the external computer 200 (see FIG. 1). The application program 1102 also receives data, sends control or configuration information, and interacts with software running on the imaging unit 400 via the communications interface 408 discussed above.

In certain embodiments, the GUI module 1104 provides a human interface for the various functions and control parameters of the system. The GUI module 1104 also displays various images, data, and notes developed or received during an imaging session.

In certain embodiments, the image processing module 1106 is responsible for “stitching” the images from the separate cameras into a single composite or mosaic retina image. For instance, in certain embodiments, eight peripheral images from the radial cameras 606 and a center image from the center camera 608 may be stitched together to form a single image of the retina up to and including the ora serrata. Additionally, the image processing module may reduce noise, if necessary, adjust exposure, format, and perform other visual enhancements to the image so that the image may be viewed on the display via the GUI module 1104.

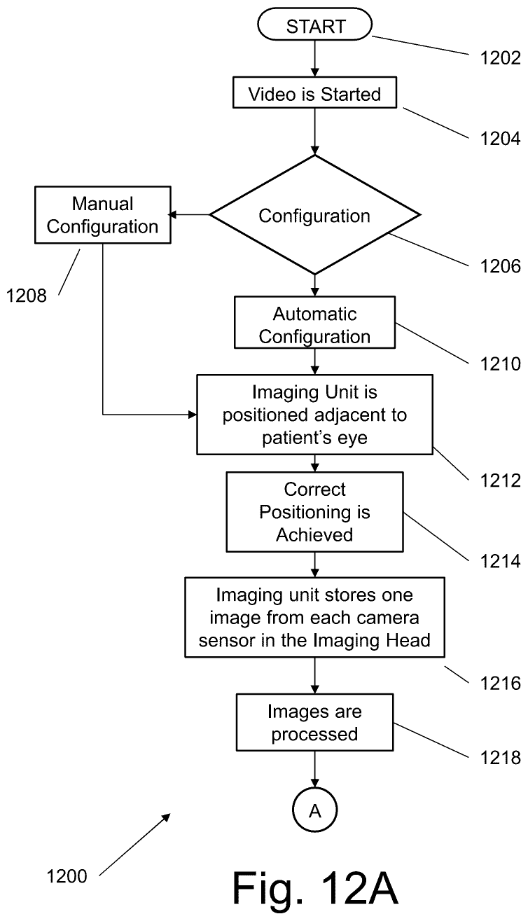

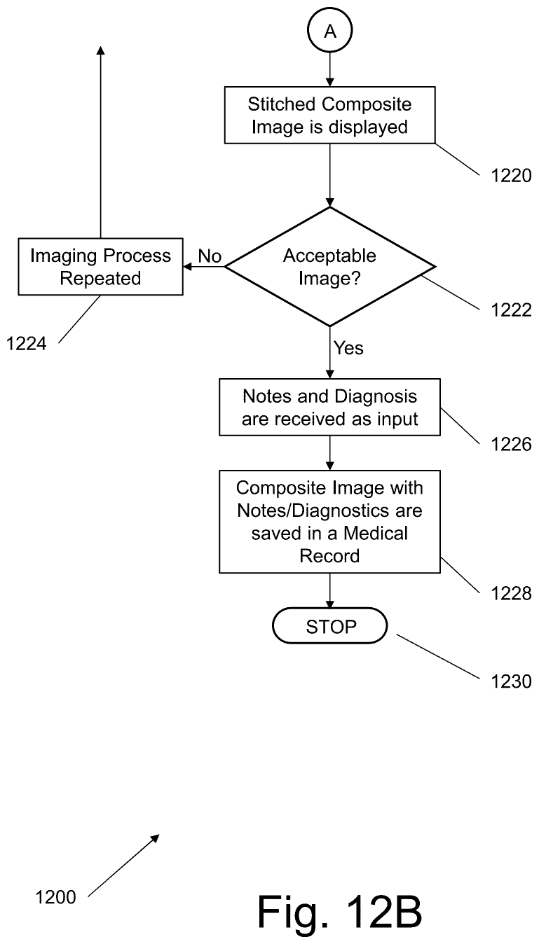

FIGS. 12A and 12B are flowcharts that illustrate an example process 1200 that may be implemented in one or more of the processors and associated memories used by the imaging system 100. The process 1200 begins at step 1202 where the imaging unit is started, and then flows to step 1204. In step 1204, a user may start a video mode for the system (either by the user interface 430 on the imaging unit or by the GUI module 1104 discussed above). In step 1206, the user may then select to manually enter certain system settings or simply opt for an automatic mode. If the user selects the manual configuration, the process flows to step 1208 where the GUI module 1104 displays a menu of system configuration settings. On the other hand, if the user selects an automatic configuration, the process flows to step 1210 where certain system settings will be set or adjusted automatically.

In step 1212, the user may position the imaging unit adjacent to the patient's eye using the coupler 500 discussed above. Once the imaging unit is adjacent to the patient's eye, the user can observe a real-time video displayed on the computer to determine if the imaging head is positioned along the centerline of the pupil of the patient's eye and in proper position. If the user determines that the imaging head 600 is properly positioned, the user may actuate the imaging unit's interface 430, which will cause the imaging unit to take a still composite photo as explained below. Alternatively, an automated fovea location recognition (AFLR) module algorithm may immediately actuate the imaging unit once the AFLR module recognizes that the imaging head 600 is in the correct position, as the user moves the imaging unit to the correct position. In certain embodiments, the real-time video stream may be from the center camera to assist with proper positioning and alignment. In other embodiments, the real-time video stream may be from a composite video produced from one or more of the radial cameras 606 in addition to the center camera 608. In yet other embodiments, a lower resolution video stream may be produced from a composite video produced by the radial cameras 606 and the center camera 608 to aid in positioning.

In either scenario, once the correct positioning has been achieved in step 1214, the imaging unit temporarily shifts to a still image mode and all the cameras or camera sensors in the camera array 605 captures and stores a still image of their respective field of view of the retina in step 1216.

The individual images are then processed in step 1218 by one or more processors discussed above to create a composite image of the retina. For instance, in one embodiment, embedded firmware or processor software in the imaging unit 400 formats and sends the individual images to the external computer 200 via the interface 408 discussed above. In one example, software running on the external computer 200 uses the individual images to create a composited stitched image of essentially the entire retina in step 1218. As discussed above, the software may also apply noise reduction and image enhancements to further refine the image for display. Note that the process 1200 shown in FIG. 12A continues to FIG. 12B via the continuation indicator “A.”

In step 1220, the image is displayed on a display of a computing device, such as the external computer 200 discussed above. In step 1222, either the user or a software module (such as an Al-enhanced diagnostic module) evaluates the composite retina image. If the user and/or the software determines that the image is not acceptable, the process flows to step 1224 which directs the process to repeat certain portions of the process as necessary until a satisfactory image can be obtained.

On the other hand, if the image is acceptable, in step 1226, the process allows for the input of a user diagnosis and other notes to be incorporated into the medical record. As discussed above, additional notes from the user may be received via a microphone integrated into the imaging unit or another input device in communication with the imaging system, such as a phone or a tablet with a microphone. After receiving the notes, in step 1228, the composite image with the notes and diagnosis may be saved in a DICOM-formatted image record, which, in some embodiments, may be stored in a secure hospital or clinic HIPAA-compliant database, and the process stops in step 1230. Once stored in the HIPAA-compliant database, the record may be available for additional review or discussion by any authorized individual.

The abstract of the disclosure is provided for the sole reason of complying with the rules requiring an abstract, which will allow a searcher to quickly ascertain the subject matter of the technical disclosure of any patent issued from this disclosure. It is submitted with the understanding that it will not be used to interpret or limit the scope or meaning of the claims.

Any advantages and benefits described may not apply to all embodiments of the invention. When the word “means” is recited in a claim element, Applicant intends for the claim element to fall under 35 USC 112(f). Often a label of one or more words precedes the word “means”. The word or words preceding the word “means” is a label intended to ease referencing of claims elements and is not intended to convey a structural limitation. Such means-plus-function claims are intended to cover not only the structures described herein for performing the function and their structural equivalents, but also equivalent structures. For example, although a nail and a screw have different structures, they are equivalent structures since they both perform the function of fastening. Claims that do not use the word “means” are not intended to fall under 35 USC 112(f).

The foregoing description of the embodiments of the invention has been presented for the purposes of illustration and description. It is not intended to be exhaustive or to limit the invention to the precise form disclosed. Many combinations, modifications, and variations are possible in light of the above teaching. For instance, in certain embodiments, each of the above-described components and features may be individually or sequentially combined with other components or features and still be within the scope of the present invention. Undescribed embodiments that have interchanged components are still within the scope of the present invention. It is intended that the scope of the invention be limited not by this detailed description, but rather by the claims.

1. A fundus camera comprising:
a housing configured for handheld operation;
an optical head disposed at a distal end of the housing, including:
a camera array including:
a plurality of radial cameras arranged around a central axis to capture retinal images of an ora serrata region of a patient's eye;
a central region camera aligned with the central axis to capture a retinal image of a center region of the eye;
a first plurality of light-emitting diodes (LEDs) assemblies positioned around the center axis and within the optical head, configured to illuminate a field of view of the plurality of radial cameras;
a second plurality of LED assemblies positioned around the center axis and within the optical head and configured to illuminate the field of view of the central region camera;
a rigid chassis positioning and supporting the camera array, the first plurality of LED assemblies, and the second plurality of LED assemblies,
a reflection control system including polarizing filters positioned to reduce glare and reflections;
a processor positioned within the housing, the processor electronically coupled to the camera array and configured to control image acquisition;
a user interface component positioned within and on the housing for actuating image capturing from the processor and the camera array; and
a communication interface coupled to the processor for transmitting image data to an external device.

2. The fundus camera of claim 1, wherein each of the plurality of radial cameras has a longitudinal optical axis which is positioned at an angle in a range of 65 to 35 degrees relative to the central axis.

3. The fundus camera of claim 1, wherein each of the plurality of radial cameras has a longitudinal optical axis which is positioned at a 50-degree angle relative to the central axis.

4. The fundus camera of claim 1, wherein the plurality of radial cameras comprises eight camera modules each including a camera sensor and camera lens.

5. The fundus camera of claim 1, wherein the plurality of radial cameras comprises camera modules having exterior dimensions smaller than 3 mm×3 mm×5 mm.

6. The fundus camera of claim 1, wherein the central region camera is a single camera module which includes a camera sensor and a camera lens centered and aligned with the central axis.

7. The fundus camera of claim 1, wherein the central region camera is a single camera module having exterior dimensions smaller than 3 mm×3 mm×5 mm.

8. The fundus camera of claim 1, wherein each of the first plurality of LEDs has a longitudinal optical axis which is positioned at an angle in a range of 65 to 35 degrees relative to the central axis.

9. The fundus camera of claim 1, wherein each of the first plurality of LEDs has a longitudinal optical axis which is positioned at a 50-degree angle relative to the central axis.

10. The fundus camera of claim 1, wherein each of the second plurality of LEDs has a longitudinal optical axis which is positioned at an angle in a range of 5 to 35 degrees relative to the central axis.

11. The fundus camera of claim 1, wherein each of the second plurality of LEDs has a longitudinal optical axis which is positioned at a 20-degree angle relative to the central axis.

12. The fundus camera of claim 1, wherein at least one of the LED assemblies in the plurality of LED assemblies comprises an LED positioned on a distal face of the chassis.

13. The fundus camera of claim 1, wherein at least one of the LED assemblies in the plurality of the LED assemblies comprises an LED and a diffuser positioned over a light path produced by the LED.

14. The fundus camera of claim 1, wherein at least one of the LED assemblies in the plurality of the LED assemblies comprises a light tube, an LED positioned at a proximal end of the light tube, and a lens positioned at the distal end of the light tube.

15. The fundus camera of claim 1, wherein at least one of the LED assemblies in the plurality of the LED assemblies comprises a light tube, an LED positioned at a proximal end of the light tube, a lens positioned at the distal end of the light tube, and a diffuser positioned over a light path produced by the LED.

16. The fundus camera of claim 1, wherein the reflection control system comprises a first plurality of polarizing filters oriented at a first angular orientation positioned with a light path of the first plurality of LEDs, a second plurality of polarizing filters oriented at the first angular orientation positioned with a light path of the second plurality of LEDs, and a third plurality of polarizing filters oriented at a second angular orientation positioned with the field of view of the cameras in the camera array to reduce reflections reaching the cameras.

17. The fundus camera of claim 1 further comprising a plurality of bridge processors to communicate with the plurality of cameras in the camera array.

18. The fundus camera of claim 17, further comprising a first printed circuit board positioned within the housing and coupled to the processor and the plurality of bridge processors.

19. The fundus camera of claim 18, further comprising a second printed circuit board positioned within the housing and in communication with the first printed circuit board, the second printed circuit board coupled to a power management system.

20.-43. (canceled)

44. A fundus camera comprising:
a housing means for handheld operation;
an optical head means disposed at a distal end of the housing, the optical head means including:
a camera array means for acquiring a plurality of images taken of separate regions of an eye;
an illumination means for illuminating a field of view of the camera array means;
a means for positioning and supporting the camera array and the illumination means;
a reflection control means for reducing glare and reflections from the eye during imaging;
a processing means for controlling image acquisition and positioned within the housing means, the processing means electronically coupled to the camera array means and;
a user interface means positioned within and on the housing means for actuating image capturing; and
a communication means coupled to the processing means for transmitting image data to an external device.

45. The fundus camera of claim 44, wherein the camera array means comprises a plurality of radial cameras arranged around a central axis to capture retinal images of peripheral regions of a patient's eye, and a central region camera aligned with the central axis to capture a retinal image of a center region of the eye.

46. The fundus camera of claim 45, wherein each of the plurality of radial cameras has a longitudinal optical axis which is positioned at an angle in a range of 65 to 35 degrees relative to the central axis.

47. The fundus camera of claim 45, wherein each of the plurality of radial cameras has a longitudinal optical axis which is positioned at a 50-degree angle relative to the central axis.

48. The fundus camera of claim 45, wherein the plurality of radial cameras comprises eight camera modules, wherein each camera module includes an image sensor and a lens.

49. The fundus camera of claim 45, wherein the central region camera is a single endoscopic camera centered and aligned with the central axis.

50. The fundus camera of claim 45, wherein the illumination means comprises a first plurality of LED assemblies orientated to illuminate a field of view of the plurality of radial cameras and a second plurality of LED assemblies orientated to illuminate a field of view of the central region camera.

51. A method of capturing a wide-field image of a retina, comprising:
positioning a handheld imaging unit adjacent to a patient's eye, the imaging unit comprising a camera array including a center camera and a plurality of radial cameras arranged about a central axis;
illuminating the patient's eye with an illumination system comprising a plurality of LED assemblies arranged to illuminate respective effective fields of view of the center camera and the radial cameras;
displaying a real-time video stream from at least one camera of the camera array on an external computing device while positioning the imaging unit; and
determining if a proper alignment has been achieved based on the real-time video stream,
if proper alignment has been achieved, initiating a capture of a plurality of still images simultaneously from the center camera and each radial camera while the imaging unit is in proper alignment with the patient's eye; and
processing the plurality of still images to generate a composite retina image having a field of view of over 180 degrees.

52. The method of claim 51, further comprising positioning a longitudinal center axis of the plurality of radial cameras at an angle in a range of 65 to 35 degrees relative to the central axis.

53. The method of claim 51, further comprising positioning a longitudinal center axis of the plurality of radial cameras at an angle of 50 degrees relative to the central axis.

54. The method of claim 51, wherein the illuminating the patient's eye further comprises illuminating outer portions of the eye with an outer plurality of LED assemblies positioned angularly between the radial cameras to illuminate the effective fields of view of the radial cameras.

55. The method of claim 51, wherein the illuminating the patient's eye further comprises illuminating an inner portion of the eye with an inner plurality of LED assemblies radially positioned below the radial cameras to illuminate the effective field of view of the center camera.

56. The method of claim 51, further comprising providing a disposable sterile coupler between the imaging unit and the patient eye, and positioning the imaging unit relative to the eye using the coupler prior to displaying the real time video stream.

57. The method of claim 51, wherein the determining if a proper alignment is achieved is performed automatically by an automated fovea location recognition (AFLR) algorithm executing on the imaging unit or external computing device.

58. The method of claim 51, wherein the processing further includes combining peripheral images from the radial cameras with the center camera image to produce a composite image of over 180 degrees of retinal coverage.

59. The method of claim 51, further comprising polarizing illumination and polarizing camera inputs using polarizing filters at the LED assemblies and at the cameras to reduce glare and reflections.

60. The method of claim 51, further comprising saving the generated composite retina image and associated user notes and diagnosis into a DICOM-formatted medical record adapted to be stored in a medical database.
